(19) United States
(12) Patent Application Publication
(10) Pub. No.: US 2025/0339703 A1
(43) Pub. Date: Nov. 6, 2025

(54) DEVICE FOR GUIDING PLASMA-JET-GENERATED SPECIES

(71) Applicant: NEOPLAS MED GMBH, Greifswald (DE)

(72) Inventor: Paul LÜTTJOHANN, Greifswald (DE)

(21) Appl. No.: 18/870,250

(22) PCT Filed: May 26, 2023
(86) PCT No.: PCT/EP2023/064167

§ 371 Date: Nov. 27, 2024

(30) Foreign Application Priority Data
May 31, 2022 (DE) 102022205543.8
May 8, 2023 (DE) 102023204245.2

(51) Int. Cl. A61N 1/44

(52) U.S. Cl. CPC A61N 1/44

(57) ABSTRACT

A device for mounting on a plasma source, with a housing that delimits a space between a plasma source and the housing. The housing has an outlet opening and an inlet opening. A sealing element is situated around the inlet opening on which the plasma source is mounted. The sealing element closes off the space between the plasma source and the inlet opening. An applicator is situated at the outlet opening of the housing. An applicator tube that is fluidically connected to the outlet opening of the housing is fastened to the proximal end of the applicator.

This document describes a device for guiding plasma jet-generated species directly to a treatment site.

### BACKGROUND

Plasma sources are being increasingly used for medical applications in the fields of human medicine as well as veterinary medicine.

A plasma is understood to mean a gas having a proportion of free electrons, radicals, ions, and neutral particles. Depending on the type of working gas, reactive species are generated by the plasma, for example reactive oxygen species such as ozone (O₃). Reactive species may have an antimicrobial effect. For this reason, treatment with plasma may assist in wound healing.

Various technologies are used in the prior art to generate medically effective plasmas. In principle, a distinction may be made between dielectric barrier discharges (DBDs) and jet plasmas.

A dielectric barrier discharge is generated by applying a high voltage between two electrodes, at least one of the electrodes being insulated by a dielectric. Generation of an arc discharge is prevented by using the insulation. Instead, a large number of fine plasma filaments generally form between the electrodes. DBDs may be generated under low pressure or also at atmospheric pressure, their special properties being realized primarily at atmospheric pressure.

The DBD may therefore be a nonthermal “cold” plasma in which the electrons have a high temperature, but the neutral gas and the ions are at approximately room temperature.

For jet plasmas, a plasma may be generated in an electromagnetic field in a discharge chamber, and in the form of a plasma jet is transported together with a gas stream from the device, in particular the discharge chamber. Application of these types of jet plasmas is described in the patent specifications DE 10 2006 019 664 A1 and EP 2 462 785 B1 by the present applicant. The cited documents describe a conveniently sized HF plasma nozzle, for which an HF matching network in the form of a separate matchbox may be dispensed with due to the specialized design. It is thus possible to implement a convenient design of the HF plasma nozzle as a handheld plasma device that can be guided manually. In a plasma treatment using a handheld plasma device, in 30 particular reactive species (radicals) and radiation (VUV/UV) which are important for the treatment effect are generated. On the other hand, the plasma is heated up due to loss processes, and the peak temperature is approximately 50° C., which under plasma conditions is regarded as “cold.”

In the case in which the surface to be treated is not the counter electrode, the dielectric barrier discharge (DBD) is relatively insensitive to uneven surfaces, but is limited to material thicknesses in the range of a few centimeters due to the available high voltages. Therefore, DBDs are often placed directly on the surface, and in many cases use the surface as a counter electrode. In contrast, jet plasmas can generally achieve an effect without direct contact with the surface. Thus, the advantage of the jet plasmas compared to the DBDs lies in their pronounced independence with respect to the surface shapes. Furthermore, jet plasmas, in contrast to DBDs, have gap accessibility; i.e., jet plasmas may also be effective directly in cavities without necessarily having to generate a saturated plasma atmosphere, as is known for DBDs.

The drawback of the two technologies is that they are both surface-based; i.e., the surface or the cavity must be directly accessible. Concealed or excessively deep treatment sites are not treatable without specific intervention, for example by exposing the treatment site.

### SUMMARY

Implementations described herein provide a device for mounting on a plasma source, which allows plasma treatment for treatment sites that previously have been difficult to access.

In some embodiments, the features of the independent claim can include such a device. Some exemplary advantageous embodiments are the subject matter of the subclaims and the following description.

Embodiments described herein facilitate positioning the device across a plasma source in such a way that the reactive species or plasma-processed gas generated by a plasma jet is intercepted and guided directly to the generated treatment site by means of an applicator tube.

Embodiments described herein may be advantageous for plasma treatment for difficult-to-access treatment sites in the fields of both veterinary medicine and human medicine.

A device according to embodiments for mounting on a plasma source includes a housing that delimits a space between a plasma source and the housing. The housing includes an outlet opening and an inlet opening. The inlet opening includes a sealing element that is configured to close off the space between the plasma source and the inlet opening. The inlet opening of the housing is provided on a plasma source. The species generated by the plasma source or the plasma-processed gas may thus pass into the space between the plasma source and the inlet opening. The housing also includes an applicator with a proximal end. The proximal end of the applicator includes an applicator tube that is fluidically connected to the outlet opening of the housing.

Advantageously, the device according to some embodiments includes the space between the plasma source and the inlet opening of the housing is closed off by a sealing element, and the plasma-processed gas or species generated by the plasma source is thus guided through the conically tapered outlet opening of the housing, via the applicator and the applicator tube, to the treatment site. The conically tapered outlet opening allows axially symmetrical bundling of the plasma-processed gas or the species generated by the plasma source prior to entry into the applicator and the applicator tube. The sealing element minimizes escape of the plasma-processed gas or the species, generated by the plasma source, between the plasma source and the inlet opening of the housing. In addition, the sealing element prevents the housing of the device from sliding from the plasma source, prior to entry into the applicator, due to back pressure from bundling of the plasma-processed gas or the species generated by the plasma source.

The plasma source may be a device for generating a cold plasma jet. The plasma source is preferably a handheld plasma device as described in the patent specifications DE 10 2006 019 664 A1 and EP 2 462 785 B1 by the present applicant and incorporated herein by reference.

The sealing element may be a sealing ring.

The sealing may be detachably fastened to the applicator. The proximal end of the applicator may include a plug connection that is suitable for connecting the applicator to the applicator tube. The plug connection may be a Luer lock connection, which is known from the prior art.

The applicator tube is configured in such a way that the reactive species generated by the plasma source may be guided to the treatment site. Deep or concealed treatment sites may be reached due to the configuration of the applicator tube. The applicator tube may be a hose or a catheter.

The object of reaching difficult-to-access treatment sites is also achieved by a device according to the invention, which does not include the above-described sealing element between the housing and the source. In other words, the sealing element is optional and may be omitted.

When the sealing element is omitted, a seal between the housing and the plasma source may be achieved by other means.

For example, the fit between the housing and the plasma source may be selected in such a way that sealing of the space between the plasma source and the housing is ensured. For example, the housing, which is mounted on the plasma source, may enclose the plasma source in the manner of a tightly fitting casing.

The fit may be selected, for example, in such a way that the housing plastically deforms when mounted on the plasma source, and thus ensures a tight connection between the housing and the plasma source. In this regard, (strong) elastic deformation of the housing is also conceivable instead of the plastic deformation.

Alternatively or additionally, an overlapping length of the housing with the plasma source may be selected to be great enough that sufficient sealing of the space within the housing is achieved.

Alternatively or additionally, the housing may be completely or partially made of a sealing material such as an elastomer, so that a separate, i.e., independent, sealing element is not necessary.

Alternatively or additionally, the plasma source may include a sealing material such as an elastomer at its outer wall, thus ensuring sealing of the space in the housing.

Even though the device according to some embodiments does not include the sealing element, it is suitable for achieving the object, in that it guides the reactive species to the treatment site by means of the applicator tube.

The applicator tube is preferably connected to the housing by means of the applicator tube fixing means. The applicator tube fixing means is more preferably used to detachably connect the applicator tube to the housing.

The applicator tube fixing means, for example as described above, may be a simple plug connection, a simple screw connection, or a Luer connection.

For the plug connection, the detachable connection between the applicator tube and the housing is ensured by frictional locking. For the plug connections with frictional locking described here, optional means for increasing the frictional locking are also generally conceivable. Such means may be rough surfaces or flutings.

For the screw connection, an internal thread that comes into detachable engagement with an external thread of the applicator tube is preferably provided at the applicator tube fixing means. However, a reverse assignment of the internal and external threads is also possible.

The Luer connection is a standardized connection, and thus allows the use of existing and/or different (standardized) cannulas, which increases the flexibility of use of the device and reduces costs. The Luer connection may be, for example, a so-called Luer slip connection (without a thread) or a so-called Luer lock connection (with a thread).

In one preferred design, an adapter is provided between the applicator tube fixing means and the applicator tube. By use of the adapter, otherwise incompatible connecting means of the applicator tube fixing means and the applicator tube may be brought into preferably detachable engagement with one another. The adapter may be used to establish a standardized connection between an unstandardized applicator tube fixing means and a standardized applicator tube (or vice versa).

When, for example, the applicator tube fixing means has a thread and the applicator tube has a plug connection, an adapter having a respective thread or a plug connection complementary thereto may be used for the preferably detachable mounting of the applicator tube at the housing of the device. This increases the flexibility and expands the range of applications for the device, since the device does not require the exchange of parts, and may be operated solely by use of the adapter with different applicator tubes.

When, for example, the applicator tube fixing means has a simple thread and the applicator tube has a Luer lock connection, an adapter having a respective thread or a Luer lock connection complementary thereto may be used for the detachable mounting of the applicator tube at the housing of the device. Correspondingly, the device may be used with standardized applicator tubes, which provides logistical and cost advantages.

When, for example, the applicator tube fixing means has a simple thread and the applicator tube has a Luer slip connection, an adapter having a respective thread or a Luer lock connection complementary thereto may be used for the detachable mounting of the applicator tube at the housing of the device. Correspondingly, the device may be used with standardized applicator tubes, which provides logistical and cost advantages.

The applicator tube may be a standardized (mass-produced) article. It may be reusable, or intended for single use as a disposable article.

The applicator tube may be, for example, an infusion tube, a cannula, or some other (medical) attachment, such as an ear attachment.

### Exemplary Embodiments

The details of one or more implementations are set forth in the accompanying drawings and the description below. Other features and advantages will be apparent from the description and drawings, and from the claims.

### DESCRIPTION OF DRAWINGS

FIG. 1 shows a schematic cross section of the device according to some embodiments.

FIG. 2 shows a schematic cross section of a further design of the device according to some embodiments.

FIG. 3 shows various variants according to some embodiments of the applicator tube fixing means and applicator tubes in a disassembled state.

FIG. 4 shows the variants according to the some embodiments of the applicator tube fixing means and applicator tubes, shown in FIG. 3, in an assembled state.

FIG. 5 shows a further example of a detachable connection between the applicator tube and the housing.

FIG. 6 shows yet a further example of a detachable connection between the applicator tube and the housing, together with a further variant of the applicator tube.

The following reference numerals are used to denote the individual elements of the design of the devices:

### LIST OF REFERENCE NUMERALS

1 applicator tube for guiding the plasma-processed gas
2 applicator tube fixing means
3 housing for bundling and guiding the plasma-processed gas
4 sealing element
5 plasma source
6 reactive species
7 plasma jet or effluent
8 adapter

### DETAILED DESCRIPTION

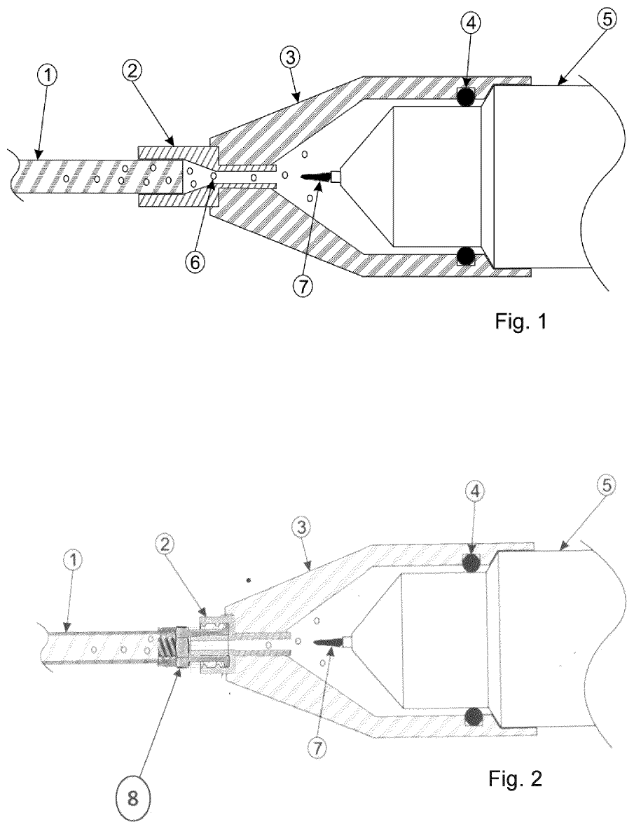

FIG. 1 shows by way of example the schematic design of the device according to some embodiments, which is mounted on a plasma jet source 5.

The plasma source 5 generates a plasma jet or effluent 7, which in turn generates reactive species 6. Due to the fact that the plasma source 5 is operated with a process gas, and the space between the plasma source 5 and the housing 3 is closed off by an optional sealing element 4, the plasma-processed gas is guided through the conically tapered outlet opening of the housing 3 via the applicator and the applicator tube 1, and preferably the applicator tube fixing means 2, to the treatment site.

The applicator tube fixing means 2 may be detachably mounted at the housing 3 via a thread, for example (not shown). In the arrangement shown, an external thread at the applicator tube fixing means 2 would be engaged with an internal thread in the housing 3. A connection via an external thread at the housing 3 and an internal thread at the applicator tube fixing means 2 is also conceivable. The detachable connection allows the applicator tube fixing means 2 to be easily exchanged if it is defective or is to be replaced by another applicator tube fixing means 2 that provides a different type of connection with the applicator tube 1.

The applicator tube fixing means 2 may also preferably be detachably connected to the housing via a simple plug connection (as shown in FIG. 1). However, a quasi-nondetachable plug connection may also be provided via a press fit or shrink fit. This type of connection is durable, and avoids the use of error-prone joining processes, and joints that may possibly deteriorate over time.

The applicator tube fixing means 2 may also be nondetachably connected to the housing via an adhesive bond or a (plastic) weld joint, for example.

The nondetachable connection between the applicator tube fixing means 2 and the housing 3 may preferably ensure a better seal than a detachable connection.

In the design shown in FIG. 1, the applicator tube 1 and the housing 3 are detachably mounted via a simple plug connection. The applicator tube 1 is plugged into the applicator tube fixing means 2 and held in the applicator tube fixing means 2 by frictional locking. Alternatively, the applicator tube I could also be attached to the applicator tube fixing means 2 from the outside.

The design shown in FIG. 2 differs from the device shown in FIG. 1 via the type of connection between the applicator tube 1 and the housing 3.

The connection between the applicator tube fixing means 2 and the housing 3 may be the same as described in conjunction with FIG. 1.

In the illustrated design, the connection between the applicator tube fixing means 2 and the applicator tube 1 takes place by means of an adapter 8. The adapter 8 is situated between the applicator tube fixing means 2 and the applicator tube 1. A Luer lock connection may thus be established between the applicator tube 1 and the applicator tube fixing means 2. In this way, the device may be used with applicator tubes that are common in medical applications, for example infusion tubes or cannulas.

Instead of the shown simple thread connection between the applicator tube fixing means 2 and the adapter 8, in the present case a plug connection, for example, would be conceivable.

In addition, the plug connection could be supplemented by a form-fit component, in which a protrusion on the adapter 8 engages with indentations in the applicator tube fixing means 2. Such a plug connection with a form fit in the sectional illustration would be similar to the simple thread shown.

A reverse arrangement of the internal and external threads (or protrusion(s) and groove(s)) at the applicator tube fixing means 2 and at the adapter 8 is likewise possible.

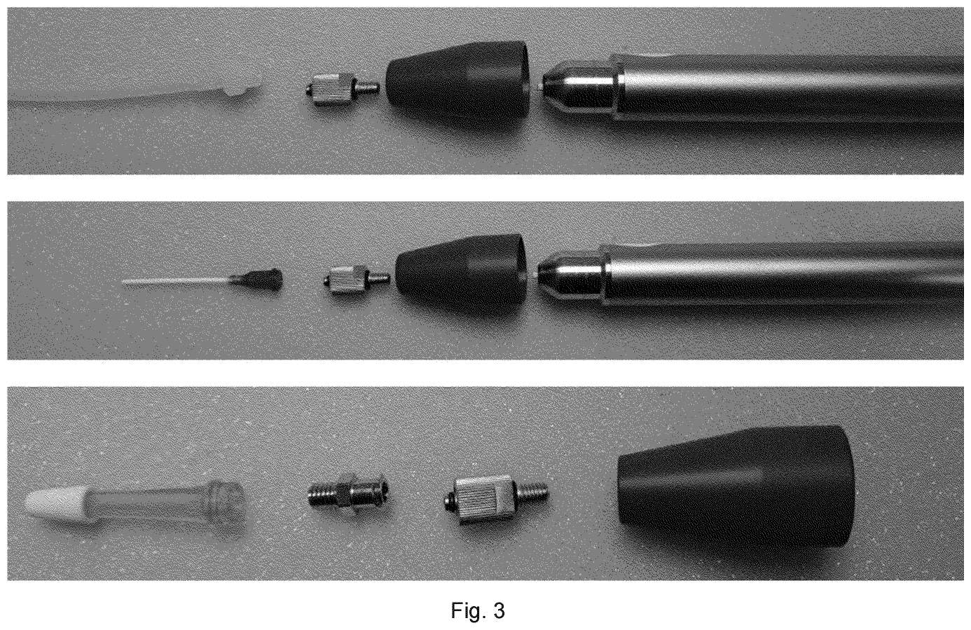

FIG. 3 shows three examples of applicator tubes 1 which are each mountable differently at the housing 3 of the device.

In the top portion of FIG. 3, the applicator tube 1 is an infusion tube with a Luer lock connection. For this purpose, the applicator tube fixing means 2 has a Luer lock connection complementary thereto, so that the infusion tube may be mounted directly at the applicator tube fixing means 2.

In the middle portion of FIG. 3, the applicator tube 1 is a cannula. In the example shown, the cannula is detachably connected to the applicator tube fixing means 2 via a simple plug connection.

In the bottom portion of FIG. 3, the applicator tube 1 is a mountable ear attachment. The applicator tube fixing means 2 is provided with a simple internal thread. In this example, the ear attachment cannot be plugged onto the applicator tube fixing means 2. Therefore, in the present case an adapter 8 is used. On one side the adapter is provided with an external thread that is compatible with the internal thread of the applicator tube fixing means 2. The ear attachment may be mounted on the other side, i.e., the other end of the adapter 8. To increase the frictional locking between the adapter 8 and the ear attachment, the adapter 8 has ring-shaped flutings at the end at which the ear attachment is to be mounted.

In all three portions of FIG. 3, a sealing element, which is designed as an O-ring by way of example, is provided at the applicator tube fixing means 2 in order to seal off the connection with the applicator tube 1 or with the adapter 8.

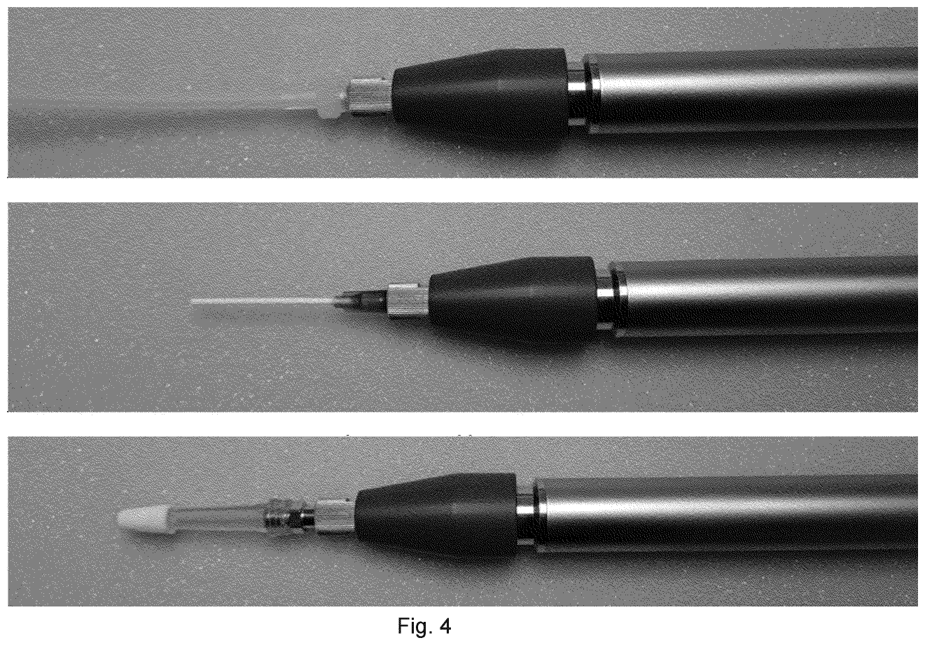

The designs of the device shown in FIG. 3 by way of example are illustrated in FIG. 4 in an assembled state. The adapter 8 connected in between, which is situated between the ear attachment and the applicator tube fixing means 2, is apparent in particular in the bottom portion of FIG. 4. In contrast, in the top and middle portions of FIG. 4 the applicator tube fixing means 2 and the infusion tube or the cannula are directly connected to one another without an adapter in between.

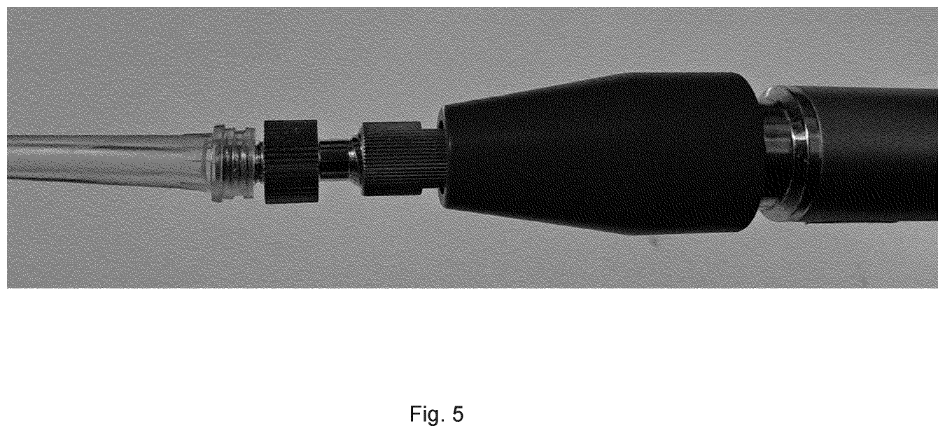

FIG. 5 shows a further design in an assembled state. In the design by way of example, an applicator tube 1 is connected to an adapter 8 via a Luer lock connection, and the adapter is in turn connected to an applicator tube fixing means 2.

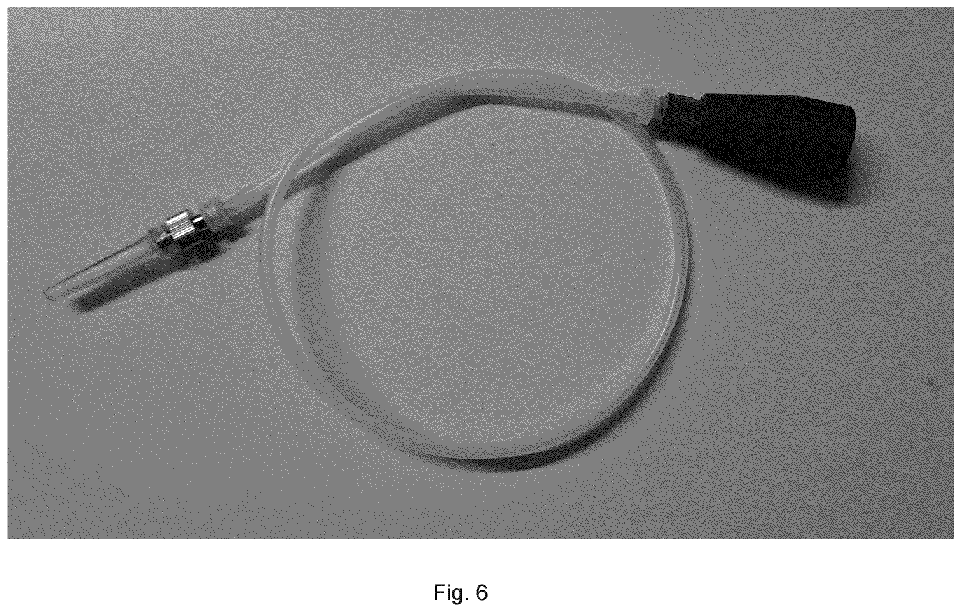

FIG. 6 shows a further design in an almost assembled state. An applicator tube 1 made up of multiple parts is screwed almost completely into the applicator tube fixing means 2 of a housing 3 by means of a Luer lock connection. The proximal portion of the applicator tube 1 is an infusion tube. The distal portion of the applicator tube 1 is connected to the infusion tube by means of an adapter, on which the applicator tube is mounted. Such combinations of assembled applicator tubes 1, which are mountable in different ways together with the applicator tube fixing means 2, and thus at the housing 3, increase the flexibility of the device according to some embodiments and allow difficult-to-access treatment sites to be reached.

1. A device for mounting on a plasma source, including a housing that delimits a space between a plasma source and the housing,
wherein the housing has an outlet opening and an inlet opening, and
wherein a sealing element is situated around the inlet opening on which the plasma source is mounted, the sealing element being configured to close off the space between the plasma source and the inlet opening; and
an applicator with a proximal end,
wherein the proximal end of the applicator includes an applicator tube fixing means that is suitable for connecting to the applicator tube, and
wherein the applicator tube is connected to the applicator tube fixing means by means of an adapter.

2. The device according to claim 1, wherein the plasma source is a device for generating a cold plasma jet, the device preferably being a handheld plasma device.

3. The device according to claim 1, wherein the sealing element (4) is a sealing ring.

4. The device according to claim 1, wherein the applicator tube is detachably fastened to the applicator.

5. (canceled)

6. The device according to claim 1, wherein the applicator tube fixing means is a Luer lock connection.

7. The device according to claim 6, wherein the applicator tube is detachably connected to the applicator tube fixing means via an the adapter.

8. The device according to claim 1, wherein the applicator tube is suitable for guiding the reactive species generated by the plasma source to the treatment site.

9. The device according to claim 8, wherein the applicator tube is configured in such a way that treatment sites can be reached.

10. The device according to claim 1, wherein the applicator tube is a hose, a cannula, or a catheter.
